(19) United States
(12) Patent Application Publication
(10) Pub. No.: US 2024/0189537 A1
(43) Pub. Date: Jun. 13, 2024

(54) BREATH REGULATING DEVICE

(71) Applicant: Samuel Meacham, Plymouth, MA (US)

(72) Inventor: Samuel Meacham, Plymouth, MA (US)

(21) Appl. No.: 18/077,586

(22) Filed: Dec. 8, 2022

(51) Int. Cl. A61M 16/20

(52) U.S. Cl. CPC A61M 16/201; A61M 2205/59

(57) ABSTRACT

A breath regulating device, including a main body, a mouthpiece disposed on at least a portion of the main body to receive air exhaled from a user therein, an air exit aperture disposed on at least a portion of the main body to facilitate extraction of the air received by the mouthpiece, and an air control dial, including a movable disc movably disposed on at least a portion of the main body to move in a first rotational direction or a second rotational direction, and a plurality of air regulating apertures disposed on at least a portion of the movable disc to control an air flow level through the air exit aperture in response to moving the movable disc, such that at least one of the plurality of air regulating apertures is disposed over the air exit aperture.

### BACKGROUND

### 1. Field

The present general inventive concept relates generally to breathing control, and particularly, to a breath regulating device.

### 2. Description of the Related Art

Many people suffer from dyspnea, which is commonly known as shortness of breath. However, other breathing difficulties include chronic obstructive pulmonary disease (COPD) and/or asthma. During such an experience, a person will often be uncomfortable due to difficulty breathing air into the lungs.

Current treatment options for these conditions usually require intake of large amounts of medication which can be incredibly costly and increase the likelihood of long-term health-related side effects.

Therefore, there is a need for a breath regulating device to relax breathing muscles of a user experiencing breathing difficulties.

### SUMMARY

The present general inventive concept provides a breath regulating device.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a breath regulating device, including a main body, a mouthpiece disposed on at least a portion of the main body to receive air exhaled from a user therein, an air exit aperture disposed on at least a portion of the main body to facilitate extraction of the air received by the mouthpiece, and an air control dial, including a movable disc movably disposed on at least a portion of the main body to move in a first rotational direction or a second rotational direction, and a plurality of air regulating apertures disposed on at least a portion of the movable disc to control an air flow level through the air exit aperture in response to moving the movable disc, such that at least one of the plurality of air regulating apertures is disposed over the air exit aperture.

The mouthpiece may glow to improve visibility in at least one of a dark condition and a low light condition.

Each of the plurality of air regulating apertures may have a different size with respect to each other.

The breath regulating device may further include a dial receiving recess disposed on at least a portion of the main body to receive the movable disc therein.

The breath regulating device may further include an air channel disposed within at least a portion of an interior of the main body to connect the mouthpiece to the air exit aperture.

The breath regulating device may further include an air valve disposed within at least a portion of the air channel to control movement of the air within the air channel.

The air valve may allow movement of the air from the mouthpiece to the air exit aperture, and prevents movement of the air from the air exit aperture to the mouthpiece.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates an exploded view of a breath regulating device, according to an exemplary embodiment of the present general inventive concept; and

FIG. 2 illustrates an isometric view of the breath regulating device, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Breath Regulating Device 100
Main Body 110
Mouthpiece 120
Air Exit Aperture 130
Air Control Dial 140
Movable Disc 141
Air Regulating Apertures 142
Dial Receiving Recess 150
Air Channel 160
Air Valve 170

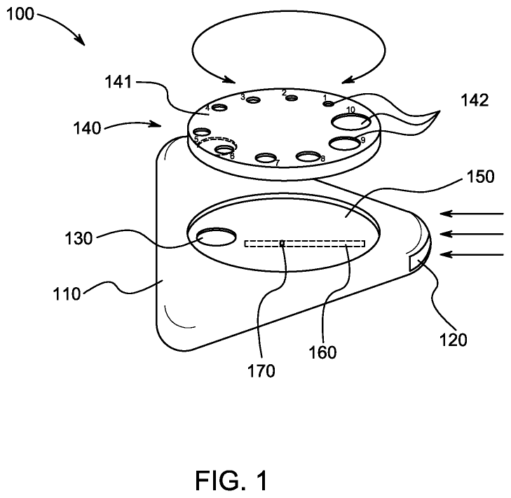

FIG. 1 illustrates an exploded view of a breath regulating device 100, according to an exemplary embodiment of the present general inventive concept.

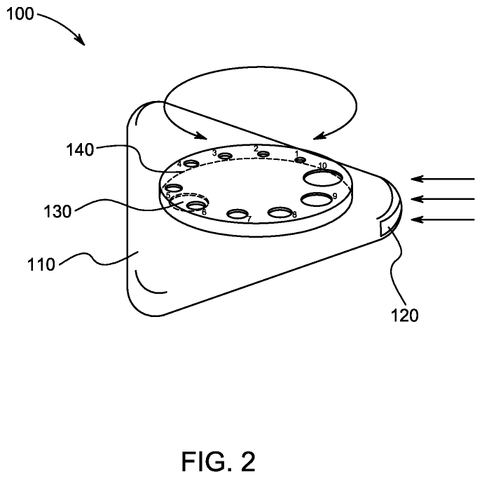

FIG. 2 illustrates an isometric view of the breath regulating device 100, according to an exemplary embodiment of the present general inventive concept.

The breath regulating device 100 may be constructed from at least one of plastic, polymer, metal, wood, and rubber, etc., but is not limited thereto.

The breath regulating device 100 may include a main body 110, a mouthpiece 120, an air exit aperture 130, an air control dial 140, a dial receiving recess 150, an air channel 160, and an air valve 170, but is not limited thereto.

Referring to FIGS. 1 and 2, the main body 110 is illustrated to have a rounded triangular shape. However, the main body 110 may be rectangular, circular, cylindrical, pentagonal, hexagonal, heptagonal, octagonal, or any other shape known to one of ordinary skill in the art, but is not limited thereto.

The main body 110 may be portable and/or handheld size. In other words, the main body 110 may have a size (i.e., a length, a width, a height) and/or a shape to fit in at least one hand of a user. The main body 110 may have instructions on use inscribed on at least a portion thereof (e.g., on a bottom surface). Also, the main body 110 may have instructions in a plurality of languages, including braille. As such, even a blind user would be able to determine how to the user the main body 110.

The mouthpiece 120 may be constructed to be softer with respect to the main body 110. For example, the mouthpiece 120 may constructed of silicone and/or a gel. More specifically, the mouthpiece 120 may at least partially deform (i.e., bend, stretch, expand) in response to an application of force (e.g., biting, pushing) thereto. As such, the mouthpiece 120 may be comfortable to the user while biting, such that the mouthpiece 120 may prevent a mouth of the user from being injured while receiving the mouth of the user thereon.

Also, the mouthpiece 120 may also be constructed of phosphors zinc sulfide and/or strontium aluminate. As such, the mouthpiece 120 may glow-in-the-dark (i.e., glows) to improve visibility by the user in a dark condition and/or a low light condition.

The mouthpiece 120 may be disposed on at least a portion of a corner of the main body 110. The mouthpiece 120 may be an aperture to receive air therein, such as while the user is breathing and/or exhaling the air into the mouthpiece 120.

The air exit aperture 130 may be disposed on at least a portion of the main body 110 and/or connected to the mouthpiece 120. The air exit aperture 130 may facilitate extraction of the air received by the mouthpiece 120.

The air control dial 140 may include a movable disc 141 and a plurality of air regulating apertures 142, but is not limited thereto.

The movable disc 141 may be movably (i.e., rotatably) disposed on at least a portion of the main body 110. The movable disc 141 may move (i.e., rotate) in a first rotational direction (i.e., clockwise) and/or a second rotational direction (i.e., counterclockwise) in response to an application of force (e.g., pushing, pulling) thereto.

The plurality of air regulating apertures 142 may be circumferentially disposed around at least a portion of a perimeter of the movable disc 141. Each of the plurality of air regulating apertures 142 may have a different size (i.e., a diameter, a circumference) with respect to each other. In other words, each of the plurality of air regulating apertures 142 have a unique size that is different from each other. For example, a first of the plurality of air regulating apertures 142 may have a first size, a second of the plurality of air regulating apertures 142 may have a second size, and a third of the plurality of air regulating apertures 142 may have a third size, such that the second size is greater than the first size, and the third size is greater than the second size.

Each of the plurality of air regulating apertures 142 may have a different size to adjust an air flow level through the air exit aperture 130. Each of the plurality of air regulating apertures 142 may be moved over the air exit aperture 130 in response to moving the movable disc 141, such that at least one of the plurality of air regulating apertures 142 may be at least partially aligned over the air exit aperture 130. As such, at least one of the plurality of air regulating apertures 142 may be selected to be used with the air exit aperture 130 according to the air flow level desired by the user. For example, the first of the plurality of air regulating apertures 142 may allow a minimum of the air flow level to move through the air exit aperture 130, and a last (e.g., a tenth) of the plurality of air regulating apertures 142 may allow a maximum of the air flow to move through the air exit aperture 130.

Accordingly, the plurality of air regulating apertures 142 may be adjusted based on how much control of the air flow level needed by the user experiencing difficulty breathing. Thus, the plurality of air regulating apertures 142 may control how slow and/or how fast the air flow level is being used to control breathing by the user, such that the user may relax.

The dial receiving recess 150 may be disposed on at least a portion of the main body 110. The dial receiving recess 150 may be recessed with respect to an outer surface of the main body 110. Moreover, the dial receiving recess 150 may receive the air control dial 140 (i.e., the movable disc 141) therein. Also, the air control dial 140 may move (i.e., rotate) within the dial receiving recess 150.

The air channel 160 may be disposed within at least a portion of an interior of the main body 110. The air channel 160 may connect the mouthpiece 120 to the air exit aperture 130. As such, the air channel 160 may facilitate movement and/or extraction of the air from the mouthpiece 120 to the air exit aperture 130.

The air valve 170 may include a one-way valve, but is not limited thereto.

The air valve 170 may be disposed within at least a portion of the air channel 160. The air valve 170 may allow movement of the air from the mouthpiece 120 to the air exit aperture 130. However, the air valve 170 may prevent movement of the air from the air exit aperture 130 to the mouthpiece 120. As such, the air valve 170 may control movement of the air within the air channel 160. Thus, the air valve 170 may facilitate breathing control to help the user relax during an episode of dyspnea, COPD, and/or asthma.

Therefore, the breath regulating device 100 may improve an ability of the user to relax while having difficulty breathing. Also, the breath regulating device 100 may be adjusted to control the air flow level as needed by the user.

The present general inventive concept may include a breath regulating device 100, including a main body 110, a mouthpiece 120 disposed on at least a portion of the main body 110 to receive air exhaled from a user therein, an air exit aperture 130 disposed on at least a portion of the main body 110 to facilitate extraction of the air received by the mouthpiece 120, and an air control dial 140, including a movable disc 141 movably disposed on at least a portion of the main body 110 to move in a first rotational direction or a second rotational direction, and a plurality of air regulating apertures 142 disposed on at least a portion of the movable disc 141 to control an air flow level through the air exit aperture 130 in response to moving the movable disc 141, such that at least one of the plurality of air regulating apertures 142 is disposed over the air exit aperture 130.

The mouthpiece 120 may glow to improve visibility in at least one of a dark condition and a low light condition.

Each of the plurality of air regulating apertures 142 may have a different size with respect to each other.

The breath regulating device 100 may further include a dial receiving recess 150 disposed on at least a portion of the main body 110 to receive the movable disc 141 therein.

The breath regulating device 100 may further include an air channel 160 disposed within at least a portion of an interior of the main body 110 to connect the mouthpiece 120 to the air exit aperture 130.

The breath regulating device 100 may further include an air valve 170 disposed within at least a portion of the air channel 160 to control movement of the air within the air channel 160.

The air valve 170 may allow movement of the air from the mouthpiece 120 to the air exit aperture 130, and prevents movement of the air from the air exit aperture 130 to the mouthpiece 120.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A breath regulating device, comprising:
a main body;
a mouthpiece disposed on at least a portion of the main body to receive air exhaled from a user therein;
an air exit aperture disposed on at least a portion of the main body to facilitate extraction of the air received by the mouthpiece; and
an air control dial, comprising:
a movable disc movably disposed on at least a portion of the main body to move in a first rotational direction or a second rotational direction, and
a plurality of air regulating apertures disposed on at least a portion of the movable disc to control an air flow level through the air exit aperture in response to moving the movable disc, such that at least one of the plurality of air regulating apertures is disposed over the air exit aperture.

2. The breath regulating device of claim 1, wherein the mouthpiece glows to improve visibility in at least one of a dark condition and a low light condition.

3. The breath regulating device of claim 1, wherein each of the plurality of air regulating apertures have a different size with respect to each other.

4. The breath regulating device of claim 1, further comprising:
a dial receiving recess disposed on at least a portion of the main body to receive the movable disc therein.

5. The breath regulating device of claim 1, further comprising:
an air channel disposed within at least a portion of an interior of the main body to connect the mouthpiece to the air exit aperture.

6. The breath regulating device of claim 5, further comprising:
an air valve disposed within at least a portion of the air channel to control movement of the air within the air channel.

7. The breath regulating device of claim 6, wherein the air valve allows movement of the air from the mouthpiece to the air exit aperture, and prevents movement of the air from the air exit aperture to the mouthpiece.
